(19) United States
(12) Patent Application Publication
(10) Pub. No.: US 2025/0025407 A1
(43) Pub. Date: Jan. 23, 2025

(54) COMPOSITION COMPRISING A COMBINATION CONSISTING OF INULIN, ALPHA-GLUCAN, HUMULUS LUPULUS EXTRACT AND LYSATE OF A FERMENTATION PRODUCT OF BACTERIA OF THE GENUS LACTOBACILLUS, USE OF THE COMPOSITION AND COSMETIC PRODUCTS COMPRISING THIS COMPOSITION

(71) Applicant: DR IRENA ERIS SPÓLKA AKCYJNA, Piaseczno (PL)

(72) Inventors: Irena SZOLOMICKA-ORFINGER, Warsaw (PL); Katarzyna ROGIEWICZ, Milanówek (PL); Renata DEBOWSKA, Minsk Mazowiecki (PL); Monika PASIKOWSKA-PIWKO, Warsaw (PL); Joanna MARKIEWICZ, Warsaw (PL)

(21) Appl. No.: 18/778,414

(22) Filed: Jul. 19, 2024

Related U.S. Application Data
(60) Provisional application No. 63/671,582, filed on Jul. 15, 2024.

(30) Foreign Application Priority Data
Jul. 20, 2023 (PL) P.445644

(51) Int. Cl. A61K 8/9789; A61K 8/06; A61K 8/73; A61K 8/99; A61Q 19/00; A61Q 19/10

(52) U.S. Cl. CPC A61K 8/9789; A61K 8/062; A61K 8/73; A61K 8/99; A61Q 19/00; A61Q 19/007; A61Q 19/10

(57) ABSTRACT

The invention relates to a composition comprising a combination of inulin, alpha-glucan, extract of common hops (Humulus lupulus) and a lysate of a fermentation product of bacteria of the Lactobacillus genus. The invention also relates to the use of the composition and to cosmetic products using the composition.

This application claims priority to Polish Patent Application No. P.445644 filed on Jul. 20,2023 and U.S. Provisional Patent Application No. 63/671,582 filed on Jul. 15, 2024, the disclosures of which are hereby incorporated by reference.

### FIELD OF THE INVENTION

The invention relates to a composition comprising a combination of inulin, alpha-glucan, extract of common hops (Humulus lupulus) and lysate of a fermentation product of bacteria of the Lactobacillus genus. The invention also relates to the use of said composition and to cosmetic products comprising it.

### STATE OF THE ART

Skin is a mechanical shield protecting the human body from external agents and pathogenic microorganisms. Its outer layer is the epidermis, which is made up mainly of corneocytes (dead cells of the stratum corneum) and keratinocytes (living cells), or in general, made up mainly of cells responsible for the synthesis of keratin and a number of protective functions. The keratinized layer of the epidermis is additionally covered by a film formed by microorganisms, which has a beneficial effect on the skin and forms a protective biological barrier that prevents the growth of harmful bacteria.

External factors such as high or low temperatures, UV radiation, bacteria or microorganisms can negatively affect both the epidermal cells, as well as the state of the resident microbiome, thereby weakening the skin's protective function and impairing its appearance. Compositions, e.g. medicinal, cosmetic or skincare products, which are rich in cosmetically active ingredients with beneficial effects, are commonly used to maintain the skin's condition.

Inulin (INCI: Inulin) is a plant-derived polysaccharide, mainly derived from chicory, with a wide range of applications in the cosmetic and food industry. As a natural prebiotic, it supports the growth of microorganisms that are beneficial to the skin, thereby strengthening its natural protective barrier. In addition, it is characterized by its antioxidant properties, thus nullifying the damaging effects of free radicals and preventing premature skin ageing. Inulin also has a moisturizing effect—it forms a protective film on the surface of the epidermis, which prevents excessive water loss.

Alpha-glucan (INCI: Alpha-Glucan Oligosaccharide) is a gluco-oligosaccharide obtained from natural sugars by enzymatic synthesis using glycosidic transferase. Like inulin, it is a prebiotic—it maintains the balance of the skin microbiome, reduces the growth of harmful organisms that cause redness and acne lesions, and moisturizes and reduces dryness of the epidermis. Among other things, alpha-glucan is added to cosmetics for sensitive and atopic skin.

Common hops (Humulus lupulus) is a climbing herbaceous plant from the Cannabaceae family, which also includes cannabis. It comprises various compounds, such as humulone, lupulone and xanthohumol, which give the plant its bitter taste. These compounds have been identified as bitter taste receptor agonists (Three TAS2R bitter taste receptors mediate the psychophysical responses to bitter compounds of hops (Humulus lupulus L.) and beer, D. Intelmann, C. Batram, C. Kuhn, G. Haseleu, W. Meyerhof, and T. Hofmann, Chemosens. Percept, vol. 2, no. 3, pp. 118-132, 2009, doi: 10.1007/s12078-009-9049-1), and thus, dried hop fruits are used to flavour and impart bitterness to beer. Hops was originally used in overseas shipments to British colonies in Asia due to its preservative properties. Nowadays, it is primarily used in beers, especially highly hopped beers or IPAS (India Pale Ale), for its characteristic bitter taste and aroma, which gives these drinks their appeal.

Common hops extract (INCI: Humulus lupulus cone extract) is extracted from the cones of the common hops plant and is characterised by a high content of, amongst others, flavonoids, terpenes, rutin and phytohormones. It exhibits antiseptic, cleansing, antibacterial and antifungal effects, especially beneficial for sensitive and reactive skin. Common hops extract is able to activate bitter taste receptors (T2R) in the skin, especially in keratinocytes and fibroblasts. This action has the effect of strengthening the function of the skin's protective barriers: the chemical barrier—by stimulating the antimicrobial response, the immune barrier—by inhibiting the inflammatory response, the physical barrier—by optimizing the extracellular matrix and improving collagen structure, and the microbiological barrier—by inhibiting the growth of harmful microorganisms.

The lysate of a fermentation product of bacteria of the Lactobacillus genus (INCI:

Lactobacillus ferment lysate) is obtained by optimized fermentation of polysaccharides with the lactic acid bacteria Lactobacillus pentosus, the strain responsible for stimulating intestinal immunity. The lysate comprises both parabiotic agents (fragments of L. pentosus obtained by lysis) and postbiotics (molecules derived from the culture supernatant). This lysate stimulates the expression of antimicrobial peptides and TLR2 receptors present in the skin, contributing to the proper functioning of the innate immunity and immune response. In addition, it protects the skin from the occurrence of inflammation and also moisturizes the skin and restores its healthy appearance. The lysate of Lactobacillus fermentation product also shows a restorative effect on the skin barrier and helps to maintain the balance and diversity of the skin microbiome.

In the state of the art, the use of inulin, alpha-glucan, common hops extract and lysate of a fermentation product of bacteria of the Lactobacillus genus as separate components of the composition, as well as the use of combinations comprising two or three of said components, is known.

CN113197837A relates to a cosmetic micro-organic composition comprising folic acid, an extract of soy fermentation product, alpha-glucan, chrysanthemum root juice, inulin, Lactobacillus bacteria and Bifidobacterium breve bacteria. According to CN113197837A, the extract of soy fermentation product may be an extract from fermentation carried out using Lactobacillus genus bacteria, and this ingredient may have a skin moisturizing effect, enhance collagen activation and revitalize skin cells.

WO2022119533A3 describes compositions for skin disinfection comprising at least one alcohol and at least one prebiotic. According to the description of the invention and the claims of WO2022119533A3, the group of prebiotics includes oligosaccharides, including alpha-glucan and inulin, as well as more than a dozen other compounds. On the other hand, prebiotics according to the invention include, inter alia, an extract of oat fermentation filtrate obtained using the Lactobacillus genus bacteria or a lysate of a fermentation product of bacteria of the Lactobacillus genus in combination with an extract of a radish root fermentation filtrate using Leuconostoc bacteria. In the embodiments, compositions comprising alpha-glucan and inulin are disclosed, and according to the claims, a composition comprising simultaneously alpha-glucan, inulin and said prebiotics is preferred.

CN110179743A relates to a skincare composition comprising, inter alia, a filtrate of a fermentation product of soya milk by Lactobacillus bacteria, inulin and alpha-glucan.

CN112826790B relates to an animal wash composition consisting of a probiotic fermented extract and an organic nourishing ingredient. The probiotic fermented extract preferably is a combination of a fermentation lysate of bacteria of the Lactobacillus genus, a fermentation lysate of bacteria of the Difibromyces genus and a fermentation product of bacteria of the Lactobacillus genus, but it can also be a single ingredient from among the above mentioned substances. According to CN112826790B, this ingredient can, among other things, exert bacteriostatic effects, form a barrier to protect the skin, enhance the metabolism of the skin layers, scavenge free radicals, activate repair mechanisms, restore balance, reduce skin inflammation and exert moisturizing effects. The organic nutrient, on the other hand, preferably is a combination of inulin, alpha-glucan, galacto-oligosaccharides and soybean oligosaccharides, while, according to the description, the organic nutrient may also represent a single ingredient among the above-mentioned compounds.

EP4134426A1 relates to a fermentation product of bacteria of the Lactobacillus genus, method for its production and its use in cosmetics and pharmaceuticals. The composition according to EP4134426A1 may comprise a number of ingredients, including, but not limited to, at least one hair growth activator, for example common hops extract listed in an extensive list of such activators. Additionally, the composition according to EP4134426A1 may comprise a cosmetic carrier, for example inulin listed in an extensive list of such carriers. EP4134426A1 also provides specific examples of cosmetic compositions-an erythema cream and an anti-wrinkle day emulsion, which comprise combinations of beta-glucan and a fermentation product of lactic acid bacteria of the Lactobacillus genus.

EP3356356B1 relates to chemical compounds with cooling properties, and compositions comprising these compounds. Among the listed ingredients of the compositions are alpha-glucan, common hops extract, inulin and a fermentation product of bacteria of the Lactobacillus genus. EP3356356B1, however, does not disclose a single composition comprising a combination of all of the mentioned ingredients, nor does it contain any suggestion to use these specific ingredients in a single composition.

In the state of the art, a four-component composition comprising inulin, alpha-glucan, common hops extract and a lysate of a fermentation product of bacteria of the Lactobacillus genus has not been described or suggested.

Unexpectedly, a combination comprising inulin, alpha-glucan, common hops extract and lysate of a fermentation product of bacteria of the Lactobacillus genus was found to have an unexpected and unpredictable technical effect, including stimulation of epidermal cell growth in the presence of inflammatory bacteria agents and improvement of the balance of the skin microbiome. This preferred technical effect was already present at very low concentrations of the components of the combination and was not observed for Biolin/P, Biotilys and Senseryn formulations, suggesting a synergistic effect resulting from the use of the subject combination of components.

The aim of the present invention is therefore to develop a composition in which it would be possible to exploit the unexpected technical effects of the four-component combination described above.

### THE SUBJECT OF THE INVENTION

The object of the invention is a composition comprising inulin, alpha-glucan, common hops extract and lysate of a fermentation product of bacteria of the Lactobacillus genus.

Preferably, the composition of the invention comprises:

| | |
|---|---|
| 0.008 to 0.8% by weight | of inulin, |
| 0.002 to 0.2% by weight | of alpha-glucan, |
| 0.00002 to 0.04% by weight | of common hops extract, |
| 0.000058 to 0.0116% by weight | of lysate of a fermentation product of |
| | bacteria of the Lactobacillus genus, |

and at least one acceptable excipient in addition to 100% by weight, the percentages being expressed based on the total weight of the composition.

In another embodiment, the composition according to the invention comprises

| | |
|---|---|
| 0.008 to 0.08% by weight | of inulin, |
| 0.002 to 0.02% by weight | of alpha-glucan, |
| 0.00002 to 0.002% by weight | of common hops extract, |
| 0.000058 to 0.0058% by weight | of lysate of a fermentation product of |
| | bacteria of the Lactobacillus genus, |

the percentages being expressed based on the total weight of the composition.

In yet another embodiment, the composition according to the invention comprises

| | |
|---|---|
| 0.08 to 0.8% by weight | of inulin, |
| 0.02 to 0.2% by weight | of alpha-glucan, |
| 0.004 to 0.04% by weight | of common hops extract, |
| 0.0029 to 0.0116% by weight | of lysate of a fermentation product of |
| | bacteria of the Lactobacillus genus, |

the percentages being expressed based on the total weight of the composition.

It is also an object of the invention to use the composition according to the invention for producing cosmetic products.

Preferably, the cosmetic product comprises from 0.05 to 4.00% by weight of the composition as defined in any of claims 1 to 4 in relation to the total weight of the cosmetic product and at least one additional cosmetically acceptable active agent and/or at least one additional cosmetically acceptable excipient.

The object of the invention is also a cosmetic product comprising the composition as defined in any of claims 1 to 4.

Preferably, the cosmetic product is in liquid, solid or semi-solid form, for example in the form of an emulsion, including an O/W emulsion and an O/W moisturizing emulsion, a gel, a foam, or a solution.

Preferably, the cosmetic product is selected from a cream, including an under-eye cream or a night cream, a day cream, a skin cleansing product, e.g. for the face, including a gel, a foam, a micellar liquid, a milk, a gel serum, a toner, preferably a face toner.

Preferably, the cosmetic product comprises

| | |
|---|---|
| 0.05 to 4.00% by weight | of the composition according to the invention, |
| 0.00 to 22.00% by weight | at least one emollient and/or wax, |
| 0.00 to 5.00% by weight | of at least one emulsifying agent, |
| 0.01 to 16.00% by weight | of at least one moisture-binding agent, |
| 0.001 to 5.00% by weight | of at least one additional cosmetically |
| | acceptable active agent, |
| 0.01 to 1.50% by weight | of at least one preservative, |
| 0.001 to 1.80% by weight | of at least one cosmetically acceptable |
| | auxiliary substance, |
| 0.00 to 3.00% by weight | of at least one consistency adjusting agent, |
| 0.00 to 0.30% by weight | of at least one fragrance composition, |
| 0.00 to 21.00% by weight | of at least one UV filter, |
| 0.00 to 14.00% by weight | of at least one surfactant, |
| up to 100% by weight | purified water. |

Preferably, the cosmetic product is in the form of an o/w moisturizing emulsion, in particular a night face cream or an eye cream comprising:

| | |
|---|---|
| 0.05 to 4.00% by weight | the composition according to the invention, |
| 5.00 to 19.00% by weight | of at least one emollient and/or wax, |
| 0.50 to 4.00% by weight | of at least one emulsifying agent, |
| 0.01 to 5.00% by weight | of at least one moisture-binding agent, |
| 0.001 to 5.00% by weight | of at least one additional cosmetically |
| | acceptable active agent, |
| 0.01 to 1.50% by weight | of at least one preservative, |
| 0.001 to 0.50% by weight | of at least one cosmetically acceptable |
| | auxiliary substance, |
| 0.01 to 1.50% by weight | of at least one consistency adjusting agent, |
| 0.01 to 0.30% by weight | of at least one fragrance composition, |
| up to 100% by weight | purified water. |

Preferably, the cosmetic product is in the form of an o/w emulsion, in particular a daily face cream comprising:

| | |
|---|---|
| 0.05 to 4.00% by weight | the composition according to the invention, |
| 5.00 to 22.00% by weight | of at least one emollient and/or wax, |
| 0.50 to 4.00% by weight | of at least one emulsifying agent, |
| 0.01 to 7.00% by weight | of at least one moisture-binding agent, |
| 1.00 to 21.00% by weight | of at least one UV filter, |
| 0.001 to 4.00% by weight | of at least one additional cosmetically |
| | acceptable active agent, |
| 0.01 to 1.50% by weight | of at least one preservative, |
| 0.001 to 1.00% by weight | of at least one cosmetically acceptable |
| 0.01 to 3.00% by weight | auxiliary substance, |
| up to 100% by weight | of at least one consistency adjusting agent, |
| | purified water. |

Preferably, the cosmetic product is in the form of a face cleansing product, in particular a gel, a foam, a micellar liquid or a gel serum for the face comprising:

| | |
|---|---|
| 0.05 to 4.00% by weight | the composition according to the invention, |
| 0.01 to 16.00% by weight | of at least one moisture-binding agent, |
| 1.00 to 14.00% by weight | of at least one surfactant, |
| 0.01 to 1.80% by weight | at least one additional cosmetically |
| | acceptable active agent, |
| 0.01 to 1.50% by weight | of at least one preservative, |
| 0.001 to 1.80% by weight | of at least one cosmetically acceptable |
| | auxiliary substance, |
| 0.01 to 0.90% by weight | of at least one consistency adjusting agent, |
| 0.01 to 0.30% by weight | of at least one fragrance composition, |
| up to 100% by weight | purified water. |

Preferably, the cosmetic product is in the form of a face cleansing product, in particular a face cleansing milk comprising:

| | |
|---|---|
| 0.05 to 4.00% by weight | the composition according to the invention, |
| 5.00 to 15.00% by weight | of at least one emollient, |
| 1.00 to 5.00% by weight | of at least one emulsifying agent, |
| 0.01 to 7.00% by weight | of at least one moisture-binding agent, |
| 0.01 to 1.00% by weight | of at least one surfactant, |
| 0.001 to 1.00% by weight | of at least one additional cosmetically |
| | acceptable active agent, |
| 0.01 to 1.50% by weight | of at least one preservative, |
| 0.001 to 0.50% by weight | of at least one cosmetically acceptable |
| | auxiliary substance, |
| 0.01 to 1.00% by weight | of at least one consistency adjusting agent, |
| up to 100% by weight | purified water. |

Preferably, the cosmetic product is in the form of a face toner comprising:

| | |
|---|---|
| 0.05 to 4.00% by weight | the composition according to the invention, |
| 0.01 to 2.00% by weight | of at least one moisture-binding agent, |
| 0.001 to 1.00% by weight | of at least one additional cosmetically |
| | acceptable active agent, |
| 0.01 to 1.50% by weight | of at least one preservative, |
| 0.01 to 1.00% by weight | of at least one cosmetically acceptable |
| | auxiliary substance, |
| up to 100% by weight | purified water. |

Preferably, the cosmetic product comprises, as at least one emollient or wax, a compound selected from the group consisting of: candelilla wax, shea butter, jojoba oil, rapeseed oil, pentaerythritol tetraisostearate, hydrogenated polydecene, oleyl erucinate, citrate of hydrogenated vegetable oil monoglycerides, vegetable oil, hydrogenated vegetable oil, mixture of esters of caprylic and capric acids with coconut oil alcohols, isopropyl isostearate, dibutyl adipate, butyl octyl salicylate, dioctyl carbonate, moringa oil, cottonseed oil.

Preferably, the cosmetic product comprises, as at least one emulsifying agent, a compound selected from the group consisting of: cetostearyl alcohol, cetostearyl alcohol polyglucoside, arachidyl alcohol, behenyl alcohol, arachidyl alcohol polyglucoside, potassium cetyl phosphate, olive oil and sorbitol fatty acid esters and olive oil and cetostearyl alcohol fatty acid esters.

Preferably, the cosmetic product comprises a compound selected from the group consisting of: glycerol, 1,2-hexanediol, propanediol, pentylene glycol.

Preferably, the cosmetic product comprises, as at least one additional active agent, a compound selected from the group consisting of: sea water, ceramides, phytosphingosine, betaine, allantoin, vitamins including vitamin C—ascorbyl tetraisopalmitate, pro-vitamin B5—D-panthenol, vitamin E—tocopherol, vitamin B3—niacinamide, and plant extracts.

Preferably, the cosmetic product comprises, as at least one preservative, a compound selected from the group consisting of: hydroxyacetophenone, gluconolactone, sodium benzoate, potassium sorbate, calcium gluconate.

Preferably, the cosmetic product comprises, as at least one cosmetically acceptable auxiliary substance, a compound selected from the group comprising pH adjusting agents, chelating agents, antioxidant agents, bulking agents, film-forming agents and opacifying powders.

Preferably, the cosmetic product comprises, as at least one consistency adjusting agent, a compound selected from the group consisting of: candelilla wax, acrylic polymers, carbomer, glycerol stearate, xanthan gum.

Preferably, the cosmetic product comprises, as at least one UV filter, a compound selected from the group consisting of: 2-[4-(diethylamino)-2-hydroxybenzoyl]benzoic acid hexyl ester, bis-ethylhexyloxyphenol-methoxyphenyl triazine, ethylhexyl triazone, methylene-bis(benzotriazolyl)-tetramethylbutylphenol.

Preferably, the cosmetic product comprises, as at least one surfactant, a compound selected from the group consisting of: a mixture of sodium salts of coconut oil fatty acids glycinates, lauryl polyglucoside, sodium salt of coconut oil fatty acids and glutamic acid, sodium lauryl glucose carboxylate, poloxamer 188, caprylic acid ester and polyglycerol-4.

### Advantages of the Invention

As mentioned above, the studies carried out have shown that the combination of inulin, alpha-glucan, common hops extract and lysate of a fermentation product of bacteria of the Lactobacillus genus allows to achieve an unexpected technical effect. This technical effect is described in detail below in the Examples.

In addition, application studies carried out using cosmetic products comprising the aforementioned four-component combination have shown that the combination provides a number of preferred effects, including, but not limited to, a skin-soothing effect, soothing skin irritation, reducing skin redness and burning, reducing skin hypersensitivity and dryness, moisturizing and nourishing the skin, reducing flakiness and skin roughness, protecting the skin from external factors and microorganisms, and the normal state of the skin microbiome.

The studies therefore confirm the preferred properties of the combination of inulin, alpha-glucan, common hops extract and lysate of a fermentation product of bacteria of the Lactobacillus genus, and the its practical applicability, for example in cosmetic products.

In addition, these cosmetic products show high stability and do not cause undesirable effects, providing effective cosmetic effect with low concentrations of cosmetically active agents, and at the same time are easy to produce without involving extraordinary technical means. The unexpected technical effects provided by the composition according to the subject invention and the products comprising it, are the result of the presence of the above mentioned four components of the composition.

### DESCRIPTION OF THE DRAWING

FIG. 1 shows the results of keratinocyte viability analyses in the presence of S. aureus bacteria for the comparative formulations.

FIG. 2 shows the results of keratinocyte viability analyses in the presence of S. aureus bacteria for the composition according to the invention.

FIG. 3 shows the results of S. aureus biofilm analyses in the presence of comparative formulations.

FIG. 4 shows the results of S. aureus biofilm analyses in the presence of the composition according to the invention. FIG. 5 shows the results of S. epidermidis biofilm analyses in comparative formulations.

FIG. 6 shows the results of S. epidermidis biofilm analyses in the presence of the composition according to the invention.

### DETAILED DESCRIPTION OF THE INVENTION

The invention relates to a composition (“composition according to the invention”) which comprises inulin, alpha-glucan, common hops extract, and lysate of a fermentation product of bacteria of the Lactobacillus genus, which are the active ingredients of the composition according to the invention.

As used in this description, the term “active ingredient” means a substance showing a biological effect, preferably cosmetic or skincare effect, particularly preferably for the skin. Preferably, “active ingredient” means “cosmetically acceptable active ingredient”.

The terms “active ingredient” or “cosmetically acceptable active ingredient” do not include ingredients of the composition according to the present invention or cosmetic products comprising the composition according to the invention, referred to as “excipients” or “cosmetically acceptable excipients”, which provide the functional characteristics of the composition, etc. stability, consistency, fragrance, etc.

The mentioned concentrations of the active ingredients of the compositions according to the invention refer to the active ingredients in their “pure” state, i.e. without any additional substances or admixtures.

In a preferred embodiment, the composition according to the invention, in addition to the active ingredients indicated above, may comprise excipients in addition to 100% by weight of the composition. In such an embodiment, the composition according to the invention preferably comprises:

| | |
|---|---|
| 0.008 to 0.8% by weight | of inulin |
| 0.002 to 0.2% by weight | of alpha-glucan |
| 0.00002 to 0.04% by weight | of common hops extract |
| 0.000058 to 0.0116% by weight | of lysate of a fermentation product of |
| | bacteria of the Lactobacillus genus, |

the percentages relating to the total weight of the composition according to the invention.

In yet further preferred embodiment, inulin may be present in a concentration of from about 0.008 to about 0.08% by weight, more preferably from 0.08 to 0.8% by weight in relation to the total weight of the composition.

Alpha-glucan may preferably be present in a concentration of from about 0.002 to about 0.02% by weight, more preferably from 0.02 to 0.2% by weight in relation to the total weight of the composition.

The common hops extract may preferably be present in a concentration of from about 0.00002 to about 0.002 weight %, preferably from 0.004 to 0.04 weight % in relation to the total weight of the composition.

The lysate of a fermentation product of bacteria of the Lactobacillus genus may preferably be present in a concentration of from about 0.000058 to about 0.00058% by weight, preferably from 0.0029 to 0.0116% by weight in relation to the total weight of the composition.

The concentrations of the active ingredients of the composition according to the invention indicated above refer to the active ingredients in their “pure” state, i.e. without any additional substances or admixtures. Obviously, mixtures of pure ingredients with excipients, for example in the form of commercially available products, can be used instead of ingredients in the pure state. In such a case, such commercially available products should be used in corresponding amounts to provide the concentrations of the individual ingredients specified above.

In the present embodiment, said excipients present in the composition according to the invention preferably comprise glycerol, propanediol, citric acid, potassium sorbate, potassium benzoate and water.

Glycerol and propanediol are used as humectants. Glycerol may preferably be present in a concentration of from about 1 to about 10% by weight, in relation to the total weight of the composition. Propanediol may preferably be present in a concentration of from about 1 to about 10% by weight, in relation to the total weight of the composition.

Citric acid, when used as a pH adjusting agent, may preferably be present in a concentration of from about 0.01 to about 0.2% by weight, in relation to the total weight of the composition.

Sodium benzoate and potassium sorbate are used as preservatives. Sodium benzoate may preferably be present in a concentration of from about 0.001 to about 0.1% by weight, in relation to the total weight of the composition. Potassium sorbate may preferably be present in a concentration of from about 0.001 to about 0.1 weight %, in relation to the total weight of the composition.

The composition according to the invention preferably constitutes a raw material that can be used in products for various purposes, preferably in skincare and cosmetic products, in particular for topical application to the human skin.

The composition may be present in such products in amounts ranging from about 0.05 to about 4.00% by weight in relation to the total weight of the product.

The composition according to the invention can be used in various product forms. Preferably, the composition according to the invention may be in a liquid, solid or semi-solid form, for example in the form of an emulsion, including an O/W emulsion and an O/W moisturizing emulsion, a gel, a foam, or a solution.

Preferably, the product comprising the composition according to the invention is a cosmetic product and is, for example, in the form of a cream, including an eye cream or a night cream, a day cream, a skin cleansing product, e.g. for the face, including a gel, a foam, a micellar liquid, a milk, a gel serum, a toner, preferably a face toner, an oil, for example a face oil.

The final form of the cosmetic product comprising the composition according to the invention depends on the cosmetically acceptable active agents and/or cosmetically acceptable excipients used. The form of the product comprising the composition according to the invention should be suitable for application at the target site of action.

Preferably, the cosmetic product comprising the composition according to the invention further comprises at least one cosmetically acceptable active agent and/or at least one additional cosmetically acceptable excipient.

The at least one additional cosmetically acceptable active agent is an agent which exhibits on its own an effect of a cosmetic or skincare character with respect to the human skin, complementing or possibly enhancing the effect of the components of the composition according to the invention described above, namely inulin, alpha-glucan, common hops extract and lysate of a fermentation product of bacteria of the Lactobacillus genus. The at least one additional cosmetically acceptable active agent may be any known agent showing a skincare or cosmetic effect on the human skin. Preferably, the additional cosmetically acceptable active agent is a substance other than the active ingredients of the composition according to the invention.

Preferably, at least one additional cosmetically acceptable active agent is selected from the group consisting of: sea water (INCI: Maris Aqua (Sea Water), (9Z)-N-[(25,3S,4R)-1,3,4-trihydroxyoctadecan-2-yl]octadec-9-enamide (INCI: Ceramide NP), 2-hydroxy-N-[(2S,3S,4R)-1,3,4-trihydroxy-2-octadecanyl]octadecanamide (INCI: Ceramide AP), 30-oxo-30-{[(2S,3S,4R)-1,3,4-trihydroxy-2-octadecanyl]amino}triacontyl-(9Z,12Z)-9,12-octadecadienoate (INCI: Ceramide EOP), phytosphingosine (INCI: Phytosphingosine), betaine (INCI: Betaine), allantoin (INCI: Allantoin), vitamins e.g. vitamin C—ascorbyl tetraisopalmitate (INCI: Tetrahexyldecyl ascorbate), pro-vitamin B5—D-panthenol (INCI: Panthenol), vitamin E—tocopherol (INCI: Tocopherol), vitamin B3—niacinamide (INCI: Niacinamide), and plant extracts.

Preferably, at least one additional cosmetically acceptable active agent may be present in the cosmetic product in a concentration of from 0.01 to 5.00% by weight in relation to the total weight of the cosmetic product according to the invention.

At least one additional cosmetically acceptable active agent may be present in the composition according to the invention alone or in the form of mixtures with other cosmetically acceptable active agents and/or cosmetically acceptable excipients to facilitate their application and/or processing into the composition, for example in the form of commercially available formulations/raw materials.

An additional cosmetically acceptable excipient is an agent that does not have an independent skincare effect, preferably on the human skin, which is used in a composition, for example to obtain a desired form of that composition or to aid or facilitate its processing. For example, additional cosmetically acceptable excipients may be:
at least one emollient and/or wax (such as, for example, candellila wax (INCI: Candellila Wax), shea butter (INCI: Shea Butter), jojoba oil (INCI: Simmondsia Chinensis Seed Oil), rapeseed oil (INCI: Canola Oil), pentaerythritol tetraisostearate (INCI: Pentaerythrityl Tetraisostearate), hydrogenated polydecene (INCI: Hydrogenated Polydecene), oleyl erucate (INCI: Oleyl Erucate), citrate of hydrogenated vegetable oil monoglycerides (INCI: Hydrogenated Vegetable Glycerides Citrate), vegetable oil (INCI: Olus Oil), hydrogenated vegetable oil (INCI: Hydrogenated Vegetable Oil), mixture of esters of caprylic and capric acids with coconut oil alcohols (INCI: Coco-Caprylate/Caprate), isopropyl isostearate (INCI: Isopropyl Isostearate), dibutyl adipate (INCI: Dibutyl Adipate), butyl acetyl salicylate (INCI: Butyl Acetyl Salicylate), dioctyl carbonate (INCI: Dicaprylyl Carbonate), moringa oil (INCI: Moringa Oleifera Oil), cottonseed oil (INCI: Gossypium (Cotton) Seed Oil)),
at least one or more emulsifying agents (such as, for example, cetostearyl alcohol (INCI: Cetearyl Alcohol), cetostearyl alcohol polyglucoside (INCI: Cetearyl Glucoside), arachidyl alcohol (INCI: Arachidyl Alcohol), behenyl alcohol (INCI: Behenyl Alcohol), arachidyl alcohol polyglucoside (INCI: Arachidyl Glucoside), potassium cetyl phosphate (INCI: Potassium Cetyl Phosphate), olive oil and sorbitol fatty acid esters (sorbitol olivate) (INCI: Sorbitan Olivate), olive oil and cetostearyl alcohol fatty acid esters (cetostearyl olivate) (INCI: Cetearyl Olivate),
at least one moisture-binding agent (humectant) (such as, for example, glycerol (INCI: Glycerin), 1,2-hexanediol (INCI: 1,2-Hexanediol), propanediol (INCI: Propanediol), pentylene glycol (INCI: Pentylene Glycol)),
at least one preservative (such as, for example, hydroxyacetophenone (INCI: Hydroxyacetophenone), gluconolactone (INCI: Gluconolactone), sodium benzoate (INCI: Sodium Benzoate), potassium sorbate (INCI: Potassium Sorbate), calcium gluconate (INCI: Calcium Gluconate)),
at least one or more auxiliary substance, selected from pH adjusting agents, chelating agents, antioxidant agents, bulking agents, film-forming agents, opacifying powders (for example, sodium carbonate (INCI: Sodium Carbonate), sodium citrate (INCI: Sodium Citrate), phytic acid sodium salt (INCI: Sodium Phytate), tocopherol (INCI: Tocopherol), citric acid (INCI: Citric Acid), microcrystalline cellulose (INCI: Microcrystalline Cellulose)), clays and silica (INCI: Silica).
at least one consistency adjusting agent (such as, for example, candellila wax (INCI: Candellila Wax), carbomer (INCI: Carbomer), glyceryl stearate (INCI: Glyceryl Stearate), xanthan gum (INCI: Xanthan Gum)),
at least one fragrance composition,
at least one UV filter (such as 2-[4-(diethylamino)-2-hydroxybenzoyl] benzoic acid hexyl ester (INCI: Diethylamino Hydroxybenzoyl Hexyl Benzoate), bis-ethylhexyloxyphenol-methoxyphenyl triazine (bemotrizinol) (INCI: Bis-Ethylhexyloxyphenol Methoxyphenyl Triazine), ethylhexyl triazone (INCI: Ethylhexyl Triazone), methylene-bis (benzotriazolyl)-tetramethylbutylphenol (bisoctrizol) (INCI: Methylene Bis-Benzotriazolyl Tetramethylbutylphenol (nano).
at least one surfactant (such as a mixture of sodium salts of coconut oil fatty acids glycinates (INCI: Sodium Cocoaphoacetate), lauryl polyglucoside (INCI: Lauryl Glucoside), sodium salt of coconut oil fatty acids and glutamic acid (INCI: Sodium Cocoyl Glutamate), sodium lauryl glucose carboxylate (INCI: Sodium Lauryl Glucose Carboxylate), poloxamer 188 (INCI: Poloxamer 188), caprylic acid and polyglycerol-4 ester (INCI: Polyglyceryl-4 Caprate))
   and water and possibly other cosmetically acceptable excipients, etc., known in the field of the invention.

A person skilled in the art will understand that the above-mentioned ingredients do not limit the scope of the invention in any way, and the above-mentioned cosmetic products may comprise both other compounds/ingredients belonging to the above-mentioned groups that are not mentioned above, as well as ingredients belonging to other groups not mentioned above typically added to the cosmetic products.

In a preferred embodiment of the invention, said cosmetic product preferably comprises:

from about 0.00 to about 22.00% by weight, of at least one
emollient and/or wax, preferably from about 5.00 to about
22.00% by weight, more preferably from about 5.00 to
about 19.00% by weight, in particular from about 5.00 to
about 15.00% by weight,
from about 0.00 to about 5.00% by weight,
of at least one emulsifying agent,
preferably from about 1.00 to about 5.00% by
weight, more preferably from about 0.50 to about
4.00% by weight,
from about 0.01 to about 16.00% by weight, of at least
one moisture-binding agent preferably from about
0.01 to about 7.00% by (humectant),
weight, more preferably from about 0.01 to about
5.00% by weight, still more preferably from about
0.01 to about 2.00% by weight,
from about 0.001 to about 5.00% by weight, of at least
one additional cosmetically preferably from about
0.001 to about 4.00% by acceptable active agent,
weight, more preferably from about 0.001 to about
1.80% by weight, for example from about 0.001 to
about 1.00% by weight,
from about 0.01 to about 1.50% by weight, of at least one
preservative, preferably from about 0.01 to about 1.00%
by weight, more preferably from about 0.01 to about
0.60% by weight,
from about 0.001 to about 1.80% by weight, of at least
one cosmetically preferably from about 0.001 to
about 1.00% by acceptable auxiliary substance,
weight, more preferably from about 0.001 to about
0.50% by weight, for example from about 0.01 to
about 1.0% by weight,
from about 0.00 to about 3.00% by weight, of at least
one consistency adjusting preferably
from about 0.01 to about 3.00% by agent,
weight, more preferably from about 0.01 to about
1.50% by weight, even more preferably from about
0.01 to about 1.00% by weight, for example from
about 0.01 to about 0.90% by weight,
from about 0.00 to about 0.30% by weight, of at least
one fragrance composition,
preferably from about 0.01 to about 0.30% by
weight, more preferably from about 0.01 to about
0.20% by weight, particularly preferably from
about 0.01 to about 0.15% by weight,
from about 0.00 to about 21.00% by weight,
of at least one UV filter
preferably from about 1.00 to about 21.00% by
weight,
from about 0.00 to about 14.00% by weight,
of at least one
surfactant
preferably from about 1.00 to about 14.00% by
(with the exception of gel serum),
weight, more preferably from about 0.01 to about
1.00% by weight,
and up to 100% by weight
of water.

As a cosmetically acceptable auxiliary substance, the cosmetic product may comprise an ingredient selected from a group comprising at least one pH adjusting agent, at least one chelating agent and at least one antioxidant agent.

At least one pH adjusting agent may preferably be present in the cosmetic product in an amount of from about 0.01 to about 0.5% by weight, in relation to the total weight of the cosmetic product.

At least one chelating agent may preferably be present in the cosmetic product in an amount of from about 0.001 to about 0.3% by weight, in relation to the total weight of the cosmetic product.

The at least one antioxidant agent may preferably be present in the cosmetic product in an amount of from about 0.01 to about 5.00% by weight, in relation to the total weight of the cosmetic product.

In addition to the specific cosmetically acceptable additional excipients belonging to the individual functional groups indicated above, the composition according to the invention may comprise other compounds or mixtures thereof belonging to the indicated functional groups, which are known in the field of the invention.

The compound used according to the invention as at least one additional cosmetically acceptable excipient is an agent known in the art, and the use of a specific chemical compound and its amount depends on the form and/or purpose and/or method of use of the particular cosmetic product comprising the composition according to the invention. The type and the amount of additional cosmetically acceptable excipients may be selected by a person skilled in the art.

At least one additional cosmetically acceptable excipient may be present in the cosmetic product comprising the composition according to the invention alone or in the form of mixtures with other active agents and/or excipients to facilitate their application and/or processing into the composition, for example in the form of commercially available formulations/raw materials.

The composition according to the invention and cosmetic products comprising the composition according to the invention can be prepared by any method known to a person skilled in the art, preferably being prepared by the methods described in the following examples.

According to the invention, the word “about”, as used herein, is to be understood as a deviation of +/−5% from the given value, reflecting inaccuracies that may occur during the course of the manufacturing of the composition according to the invention, e.g. during the measuring of the ingredients of the composition according to the invention.

### EXAMPLES

### Example 1: Compositions According to the Invention

The following were used as raw materials for the compositions:
BIOLIN/P (Ultra Chemical Inc.)—a formulation consisting of approximately 80% by weight inulin and approximately 20% by weight alpha-glucan,
SENSERYN (Provital)—a formulation consisting of a 2% by weight common hops extract and auxiliary ingredients, including 57.8% by weight propanediol, 40% by weight glycerol and 0.2% by weight citric acid.
BIOTILYS (Greentech)—a formulation comprising lysate of a fermentation product of bacteria of the Lactobacillus genus at a concentration of 5.8% by weight and auxiliary ingredients, including 0.3% by weight sodium benzoate and 0.15% by weight potassium sorbate, and up to 100% by weight water.

The indicated raw materials were mixed together to produce four compositions as follows:

| | Com- | Com- | Com- | Com- |
|---|---|---|---|---|
| | position | position | position | position |
| | 1A | 1B | 1C | 1D |
| | [% by | [% by | [% by | [% by |
| | weight] | weight] | weight] | weight] |
| BIOLIN/P | 0.1 | 0.01 | 0.1 | 0.1 |
| BIOTILYS | 0.001 | 0.001 | 0.001 | 0.01 |
| SENSERIN | 0.001 | 0.001 | 0.01 | 0.1 |

Which, when converted to the content of pure active ingredients, yields the following compositions:

| | Com- | Com- | Com- | Com- |
|---|---|---|---|---|
| | position | position | position | position |
| | 1A | 1B | 1C | 1D |
| | [% by | [% by | [% by | [% by |
| | weight] | weight] | weight] | weight] |
| Inulin | 0.08 | 0.008 | 0.08 | 0.08 |
| (INCI: Inulin) | | | | |
| Alpha-glucan | 0.02 | 0.002 | 0.02 | 0.02 |
| (INCI: Alpha-Glucan | | | | |
| Oligosaccharide) | | | | |
| lysate of a fermentation | 0.000058 | 0.000058 | 0.000058 | 0.00058 |
| product of bacteria of | | | | |
| the Lactobacillus genus | | | | |
| (INCI: Lactobacillus | | | | |
| ferment lysate) | | | | |
| Commons hops extract | 0.00002 | 0.00002 | 0.0002 | 0.002 |
| (INCI: Humulus Lupulus | | | | |
| cone extract) | | | | |

### Example 2: In Vitro Assays-Activity of the Composition According to the Invention

The compositions of Example 1 were subjected to assays comparing the activity of the composition according to the invention against its individual components. The aforementioned commercially available formulations BIOLIN/P (Ultra Chemical Inc.), SENSERYN (Provital) and BIOTILYS (Greentech) were used as reference compositions.

### Example 2.1: Analysis of Keratinocyte Viability in the Presence of Staphylococcus aureus Bacteria

### Materials and Methods:

Human keratinocytes of the HaCaT line were seeded in a 12-well plate at 4.0×10⁴/well on 1.5 cm diameter sterile round coverslips placed inside wells in DMEM medium (Gibco) comprising 4.5 g/L glucose and 10% bovine serum without the addition of antibiotics. HaCaT cells were cultured in an incubator for 4-5 days at 37° C., 95% humidity and 5% CO₂. After this period, the culture medium was replaced with culture medium supplemented with Staphylococcus aureus ATCC 6538 (Sa) standard strains at 100 CFU/well of the analyzed substances: Biolin/P (P), Biotilys (B), Senseryn(S) formulations at respective concentration (0.001%, 0.01% and 0.1%) and compositions of Example 1: 1A (A), 1B (B), 1C (C) and 1D (D). The control samples in the assay consisted of cells treated with nothing (K), cells treated only with bacteria without test substances (K Sa) and cells treated with substances in corresponding concentrations without bacteria (K P1, K P2, K P3, KA, K B, K C, K D). The systems thus prepared were incubated at 37° C., 95% humidity and 5% CO2 for 18 hours. After this time, medium was removed from the wells and the cells were stained for 15 minutes with fluorescent dyes: SYTO 9 and propidium iodide (PI) (LIVE/DEAD BacLight™ Bacterial viability Kit reagent, ThermoFisher Scientific), diluted with buffered saline solution to a final concentration of 2.783 pM. SYTO 9 dye applied alone labels DNA in bacteria and eukaryotic cells—both with intact membranes and with damaged membranes. Propidium iodide penetrates only cells with damaged membranes, causing a reduction in the fluorescence of the SYTO 9 dye when both dyes are present. After staining, slides were fixed with 4% formaldehyde solution for 10 min, after which formaldehyde was replaced with buffered saline solution. Imaging was performed with a Leica TCS SP8 confocal microscope using a 20×/0.75NA dry objective and two lasers at 488 nm (SYTO 9 excitation) and 552 nm (PI excitation). Fluorescence emission was recorded sequentially on two detectors in the ranges 495-537 nm (SYTO 9) and 573-674 nm (PI). Single optical planes were collected at 0.684 pm intervals and the imaging volume was 6-25 pm. 6 to 20 representative fields of view from two slides were imaged for a given experimental condition. Microscopic images were analyzed by Image software. The three-dimensional volume was first reduced to two-dimensional images using an algorithm that creates a projection/photo comprising the pixels with the highest signal intensity (“maximum intensity projection”). Dye fluorescence intensity was measured in the nuclear areas of the keratinocytes. Overlapping areas of much more intense signal from bacteria were removed using mathematical operations on the images. An average value of the fluorescence intensity of a given dye was obtained for each field of view. In addition, the ratio of SYTO 9 signal intensity to PI signal intensity was measured for individual pixels by performing a split operation between channels for individual fields of view. Statistical analysis of the results was performed in GraphPad Prism 6 software.

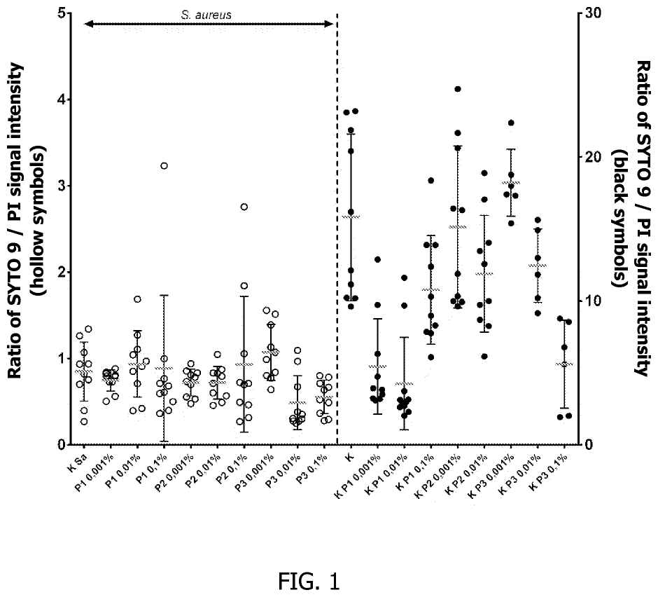

The results of keratinocyte viability analyses in the presence of S. aureus bacteria for reference compositions comprising the specified active ingredient are shown in FIG. 1.

The markings on the X axis indicate:
K Sa HaCaT cells+S. aureus bacteria
P1 Biolin/P
P2 Biotilys
P3 Senseryn
K control of HaCaT cell viability without bacteria and formulations
K P1 Control with Biolin/P
KP2 Control with Biotilys
K P3 Control with Senseryn

Symbols represent mean values for keratinocytes from the entire single field of view; horizontal bold lines indicate mean values for all fields analyzed; whiskers represent standard deviation. Blank symbols refer to keratinocytes treated with bacteria (left Y axis); black symbols refer to keratinocytes without contact with bacteria (right Y axis).

TABLE 1
Statistical analysis by Kruskal-Wallis test (multiple group comparisons)
or Mann-Whitney test (two-group comparisons)

| | | | |
|---|---|---|---|
| K Sa vs P1 0.001% | ns | K vs K P1 0.001% | * |
| K Sa vs P1 0.01% | ns | K vs K P1 0.01% | *** |
| K Sa vs P1 0.1% | ns | K vs K P1 0.1% | ns |
| K Sa vs P2 0.001% | ns | K vs K P2 0.001% | ns |
| K Sa vs P2 0.01% | ns | K vs K P2 0.01% | ns |
| K Sa vs P2 0.1% | ns | K vs K P3 0.001% | ns |
| K Sa vs P3 0.001% | ns | K vs K P3 0.01% | ns |
| K Sa vs P3 0.01% | ns | K vs K P3 0.1% | * |
| K Sa vs P3 0.1% | ns | K P1 0.001% vs K P1 0.01% | ns |
| P1 0.001% vs P1 0.01% | ns | K P1 0.001% vs K P1 0.1% | ns |
| P1 0.001% vs P1 0.1% | ns | K P1 0.001% vs K P2 0.001% | * |
| P1 0.001% vs P2 0.001% | ns | K P1 0.001% vs K P2 0.01% | ns |
| P1 0.001% vs P2 0.01% | ns | K P1 0.001% vs K P3 0.001% | ** |
| P1 0.001% vs P2 0.1% | ns | K P1 0.001% vs K P3 0.01% | ns |
| P1 0.001% vs P3 0.001% | ns | K P1 0.001% vs K P3 0.1% | ns |
| P1 0.001% vs P3 0.01% | ns | K P1 0.01% vs K P1 0.1% | ns |
| P1 0.001% vs P3 0.1% | su | K P1 0.01% vs K P2 0.001% | *** |
| P1 0.01% vs P1 0.1% | ns | K P1 0.01% vs K P2 0.01% | ns |
| P1 0.01% vs P2 0.001% | ns | K P1 0.01% vs K P3 0.001% | *** |
| P1 0.01% vs P2 0.01% | ns | K P1 0.01% vs K P3 0.01% | ns |
| P1 0.01% vs P2 0.1% | ns | K P1 0.01% vs K P3 0.1% | ns |
| P1 0.01% vs P3 0.001% | ns | K P1 0.1% vs K P2 0.001% | ns |
| P1 0.01% vs P3 0.01% | ns | K P1 0.1% vs K P2 0.01% | ns |
| P1 0.01% vs P3 0.1% | ns | K P1 0.1% vs K P3 0.001% | ns |
| P1 0.1% vs P2 0.001% | ns | K P1 0.1% vs K P3 0.01% | ns |
| P1 0.1% vs P2 0.01% | ns | K P1 0.1% vs K P3 0.1% | ns |
| P1 0.1% vs P2 0.1% | ns | K P2 0.001% vs K P2 0.01% | ns |
| P1 0.1% vs P3 0.001% | ns | K P2 0.001% vs K P3 0.001% | ns |
| P1 0.1% vs P3 0.01% | ns | K P2 0.001% vs K P3 0.01% | ns |
| P1 0.1% vs P3 0.1% | ns | K P2 0.001% vs K P3 0.1% | * |
| P2 0.001% vs P2 0.01% | ns | K P2 0.01% vs K P3 0.001% | ns |
| P2 0.001% vs P2 0.1% | ns | K P2 0.01% vs K P3 0.01% | ns |
| P2 0.001% vs P3 0.001% | ns | K P2 0.01% vs K P3 0.1% | ns |
| P2 0.001% vs P3 0.01% | ns | K P3 0.001% vs K P3 0.01% | ns |
| P2 0.001% vs P3 0.1% | ns | K P3 0.001% vs K P3 0.1% | ** |
| P2 0.01% vs P2 0.1% | ns | K P3 0.01% vs K P3 0.1% | ns |
| P2 0.01% vs P3 0.001% | ns | K Sa vs K Mann-Whitney test | **** |
| P2 0.01% vs P3 0.01% | ns | | |
| P2 0.01% vs P3 0.1% | ns | | |
| P2 0.1% vs P3 0.001% | ns | | |
| P2 0.1% vs P3 0.01% | ns | | |
| P2 0.1% vs P3 0.1% | ns | | |
| P3 0.001% vs P3 0.01% | ** | | |
| P3 0.001% vs P3 0.1% | * | | |
| P3 0.01% vs P3 0.1% | su | | |

(* p < 0.05;
** p < 0.01; p < 0.001; p < 0.0001;
ns—notsignificant):

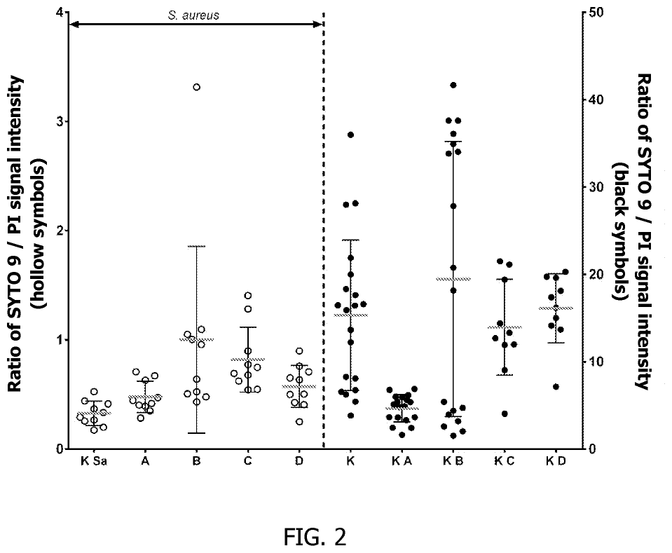

The results of keratinocyte viability analyses in the presence of S. aureus bacteria for the composition according to the invention are shown in FIG. 2. The markings on the X axis indicate:
K Sa HaCaT cells +S. aureus bacteria
A Composition 1A of Example 1
B Composition 1B of Example 1
C Composition 1C of Example 1
D Composition 1D of Example 1
K Control of HaCaT cell viability without bacteria and formulations
KA Control +Composition 1A of Example 1
KB Control +Composition 1B of Example 1
KC Control +Composition 1C of Example 1
KD Control +Composition 1D of Example 1

Symbols represent mean values for keratinocytes from the entire single field of view; horizontal bold lines indicate the mean value for all fields analyzed; whiskers represent the standard deviation. Blank symbols refer to keratinocytes treated with bacteria and formulations (left Y axis); black symbols refer to keratinocytes without contact with bacteria (right Y axis).

TABLE 2
Statistical analysis by Kruskal-Wallis test (multiple_group
comparisons) or Mann-Whitney test (two-group comparisons)

| | | | |
|---|---|---|---|
| K Sa vs Composition 1A | ns | K vs control + Composition 1A | *** |
| K Sa vs Composition 1B | *** | K vs control + Composition 1B | ns |
| K Sa vs Composition 1C | | K vs control + Composition 1C | ns |
| K Sa vs Composition 1D | ns | K vs control + Composition 1D | ns |
| Composition 1A vs | ns | Control + Composition 1A vs. | ** |
| Composition 1B | | control + Composition 1B | |
| Composition 1A vs | ns | Control + Composition 1A vs. | * |
| Composition 1C | | control + Composition 1C | |
| Composition 1A vs | ns | Control + Composition 1A vs. | ** |
| Composition 1D | | control + Composition 1D | |
| Composition 1B vs | ns | Control + Composition 1B vs. | ns |
| Composition 1C | | control + Composition 1C | |
| Composition 1B vs | ns | Control + Composition 1B vs. | ns |
| Composition 1D | | control + Composition 1D | |
| Composition 1C vs | ns | Control + Composition 1C vs. | ns |
| Composition 1D | | control + Composition 1D | |
| K Sa vs K Mann- | **** | | |
| Whitney test | | | |

(* p < 0.05;
** p < 0.01; p < 0.001; p < 0.0001;
ns—notsignificant):

Summary of results:

The different concentrations of the active ingredient in Biolin/P, Biotilys and Senseryn formulations have no effect on the increase in keratinocyte viability when combined with S. aureus bacteria compared to the control (K Sa). Only the mixture of inulin and alpha-glucan (Biolin/P) and the lysate of a fermentation product of bacteria of the Lactobacillus genus (Biotilys) at a concentration of 0.1% by weight reduced keratinocyte viability. No effect of the active ingredients on epidermal cell growth was observed in the presence of S. aureus.

Analysis of the viability of keratinocytes cultured in the presence of S. aureus and all four compositions of Example 1, unexpectedly shows a stimulation of cell growth of the HaCaT lineage compared to the control sample (K Sa). For the compositions marked as 1B and 1C, this result was statistically significant.

The Biolin/P, Biotilys and Senseryn formulations used in the presence of S. aureus had no effect on epidermal cell proliferation (FIG. 1), while the compositions according to the invention promoted HaCaT growth in the presence of bacterial infection (FIG. 2).

Furthermore, Composition 1A when used alone without bacterial superinfection inhibited epidermal cell growth, while in contact with S. aureus it unexpectedly stimulated HaCaT growth (FIG. 2).

### Example 2.2: Study of the Effect of Emulsion on Biofilm Formation on Keratinocyte Monolayers With Quantification of the Number of Bacteria by Quantitative Culture

### Materials and Methods:

Human keratinocytes of the HaCaT line were seeded in a 96-well plate at 4.0×10³/well in DMEM medium (Gibco) comprising 4.5 g/L glucose and 10% bovine serum without the addition of antibiotics. HaCaT cells were cultured in an incubator for 4-5 days at 37° C., 95% humidity and 5% CO₂. After this period, the culture medium was removed and replaced with fresh medium comprising Staphylococcus aureus ATCC 6538 or Staphylococcus epidermidis ATCC 14990 standard strains, at 3.8×10 CFU/well, and the analyzed substances. The control samples in the test were keratinocyte cells inoculated with Staphylococcus epidermidis or Staphylococcus aureus, respectively, at 3.8×jQ4 CFU/well, with culture medium (DMEM with 4.5 g/L glucose, 10% bovine serum), without the addition of test substances. Assays with both test and control samples were performed in duplicate 6 times. The systems thus prepared were incubated at 37° C., 95% humidity and 5% CO₂, for 18 hours. After this time, the planktonic forms of the bacteria were washed off, the resulting biofilm was eradicated using shaking with 0.5% saponin solution and a series of dilutions were performed. After 24 h of incubation at 37° C., the grown colonies were counted. Statistical analysis of the results was performed in GraphPad Prism 6 software, using the Mann-Whitney U statistical test (comparison of two groups).

### Example 2.2.1: Results of Staphylococcus aureus Biofilm Analysis

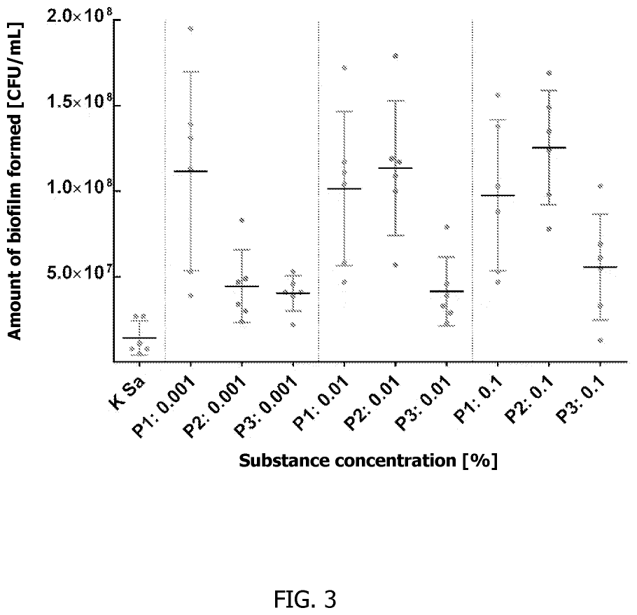

The results of the analysis of S. aureus biofilm in the presence of Biolin/P, Biotilys and Senseryn formulations are shown in FIG. 3. (K Sa—Staphylococcus aureus biofilm control on keratinocyte monolayer, in medium without the addition of test substances, other X-axis markings have the same meaning as for FIG. 1)

The graph shows the amounts of biofilm formed, obtained in several replicates of a given dilution (points), the mean of the obtained results (horizontal black line) and the standard deviation (whiskers). All samples at each concentration tested show a statistically significant increase in S. aureus biofilm formation compared to the control sample (K Sa) (p<0.05).

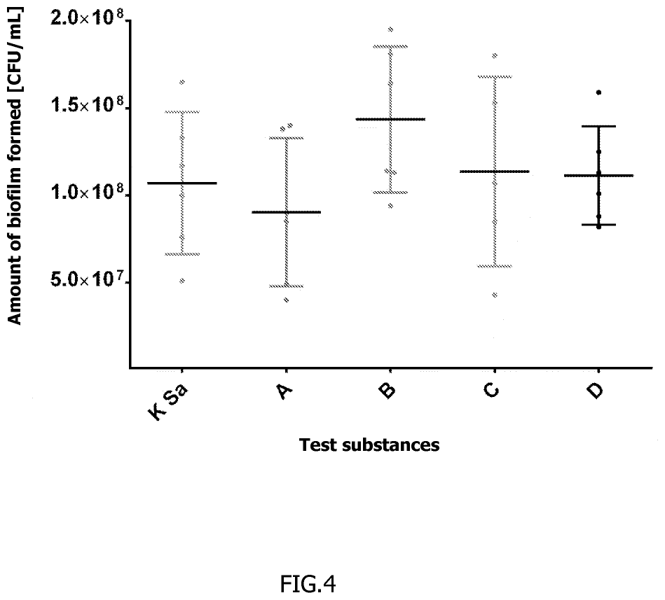

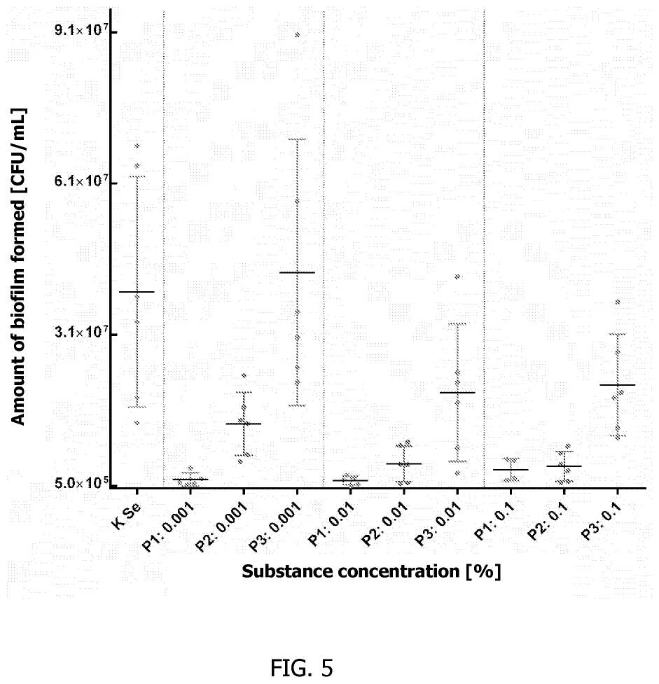

The results of the analyses of the S. aureus biofilm in the presence of the composition of Example 1 are shown in FIG. 4 (K Sa—Staphylococcus aureus biofilm control on a keratinocyte monolayer, in medium without the addition of the test substances, other X-axis markings have the same meaning as for FIG. 2). The graph shows the amounts of biofilm formed, obtained in several replicates of a given dilution (points), the mean of the obtained results (horizontal black line) and the standard deviation (whiskers). A slight inhibition of biofilm formation is observed for composition 1A, but it is not statistically significant compared to the control sample. The presence of composition 1B slightly increases the amount of S. aureus biofilm formation, but the result is not statistically significant (p>0.05). Compositions 1C and 1D have no effect on S. aureus biofilm formation (after 18 h of incubation).

### Summary of Results:

The presence of each of the Biolin/P, Biotilys and Senseryn formulations tested statistically significantly increased the amount of S. aureus biofilm formed. Biolin/P substance (0.001%) had the strongest effect on biofilm formation, followed by Biotilys (0.01% and 0.1%), and Senseryn had the weakest effect (at each concentration).

Creating the conditions for S. aureus biofilm formation is detrimental for health. In atopic dermatitis, excessive skin colonization by S. aureus promotes exacerbations of the disease and the formation of superinfections (Amy S. Paller et al: The microbiome in patients with atopic dermatitis. J Allergy Clin Immunol 2019; 143:26-35.).

A slight inhibition of biofilm formation is observed with composition 1A. Composition B slightly increases the amount of S. aureus biofilm formed. Compositions 1C and 1D have no effect on S. aureus biofilm formation.

Unexpectedly, the combination of active ingredients in the tested compositions 1A, 1B, C, D did not affect S. aureus biofilm growth, as observed when Biolin/P, Biotilys and Senseryn formulations were used (FIG. 3).

The use of compositions 1A, 1B, 1C, 1D shows the benefit of maintaining the balance of the epidermal microbiome in the presence of S. aureus.

### Example 2.2.2: Results of Staphylococcus epidermidis Bacteria Biofilm Analysis

The results of the analyses of the S. epidermidis biofilm in the presence of Biolin/P, Biotilys and Senseryn formulations are shown in FIG. 5. The graph shows the amounts of biofilm formed, obtained in several replicates of a given dilution (points), the mean of the obtained results (horizontal black line) and the standard deviation (whiskers). Biolin/P shows an inhibitory effect on S. epidermidis biofilm formation (p<0.05) at all concentrations used. Biotilys also shows an inhibitory effect, lower than Biolin/P, but still statistically significant (p<0.05). Senserin does not show a statistically significant difference in inhibiting biofilm formation (p<0.05), but the resulting mean biofilm amounts at 0.1 wt. % and 0.01 wt. % concentrations are lower than for the control sample, as shown in Table 3.

TABLE 3
S. epidermidis bacteria biofilm characteristics for single-component
compositions comprising a specific active ingredient

| | | Mean amount of | |
|---|---|---|---|
| | | bacteria obtained | p-value (control |
| | Tested | from S. | sample vs test |
| | concen- | epidermidis | sample with S. |
| Test sample | tration | biofilm [CFU/mL] | epidermidis) |
| Control | — | 3.9 × 10\hat{ }7 | — |
| Biolin/P (P | 0.1% | 3.7 × 10\hat{ }6 | 0.0095* |
| | 0.01% | 1.5 × 10\hat{ }6 | 0.0043* |
| | 0.001% | 1.3 × 10\hat{ }6 | 0.0043* |
| Biotylis (B) | 0.1% | 4.4 × 10\hat{ }6 | 0.0022* |
| | 0.01% | 4.9 × 10\hat{ }6 | 0.0022* |
| | 0.001% | 1.3 × 10\hat{ }7 | 0.0260* |
| Senserin (S) | 0.1% | 2.1 × 10\hat{ }7 | 0.1429^{$} |
| | 0.01% | 1.9 × 10\hat{ }7 | 0.1797^{$} |
| | 0.001% | 4.3 × 10\hat{ }7 | 0.9372^{$} |

*statistically significant difference (p < 0.05)
^{$}difference not statistically significant (p > 0.05)

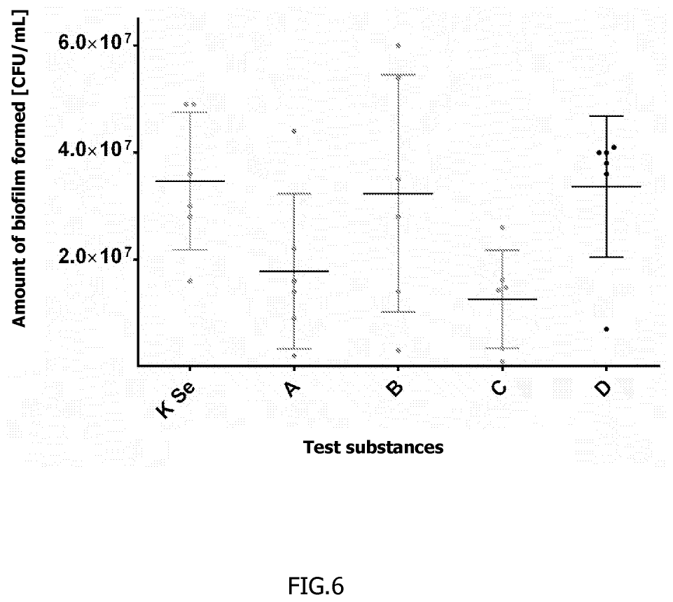

The results of the analyses of S. epidermidis biofilm in the presence of the compositions of Example 1 are shown in FIG. 6. The graph shows the amounts of biofilm formed, obtained in several replicates of a given dilution (points), the mean of the obtained results (horizontal black line) and the standard deviation (whiskers). Compositions 1A and 1C show a statistically significant ability to inhibit S. epidermidis biofilm formation, while compositions 1B and 1D show no such effect compared to the control sample. In the case of compositions 1B and 1D, the amount of biofilm formed is similar to that obtained for the control sample. Table 4 shows the average amount of bacteria obtained from the S. epidermidis biofilm after 18 h of incubation with compositions A, B, C and D.

TABLE 4
S. epidermidis bacteria biofilm characteristics for
compositions 1A, 1B, 1C and 1D of Example 1.

| | Mean amount of bacteria | p-value (control |
|---|---|---|
| | isolated from S. | sample vs |
| Tested | epidermidis biofilm | test sample with |
| substance | [CFU/mL] | S. epidermidis) |
| Control | 3.8 × 10\hat{ }7 | — |
| A | 2.4 × 10\hat{ }7 | 0.0476* |
| B | 4.4 × 10\hat{ }7 | 0.8463^{$} |
| C | 1.8 × 10\hat{ }7 | 0.0087* |
| D | 3.9 × 10\hat{ }7 | 0.8528^{$} |

*statistically significant difference (p < 0.05)
^{$}difference not statistically significant (p > 0.05)

### Summary of Results:

A slight inhibition of S. aureus biofilm formation is observed with composition 1A. Composition 1B slightly increases the amount of S. aureus biofilm formation. Compositions 1C, 1D have no effect on S. aureus biofilm formation.

Unexpectedly, the combination of substances in tested compositions 1A, 1B, 1C and 1D did not affect the growth of S. aureus biofilm, as was observed when Biolin/P, Biotilys and Senseryn formulations were used (FIG. 3).

The use of compositions 1A, 1B, 1C and 1D shows the benefit of maintaining the balance of the epidermal microbiome in the presence of S. aureus.

Compositions 1A, 1B, 1C and 1D of Example 1 reduced the growth of S. epidermidis biofilm to a lesser extent than the Biolin/P, Biotilys and Senseryn formulations.

Inhibition of S. epidermidis biofilm growth (FIG. 5) while promoting S. aureus biofilm growth (FIG. 3) is detrimental to the proper balance of the epidermal microbiome in dermatoses (Koh et al.: Skin microbiome of atopic dermatitis. Allergology International 71 (2022)).

### Final Conclusions:

Compositions 1A, 1B, 1C and 1D of Example 1 stimulated epidermal cell growth in the presence of S. aureus infection, which was not observed for Biolin/P, Biotilys and Senseryn formulations;
Compositions 1A, 1B, 1C and 1D of Example 1 did not induce S. aureus biofilm growth, whereas Biolin/P, Biotilys and Senseryn formulations stimulated the growth of these bacteria. As mentioned above, creating the conditions for S. aureus biofilm formation is detrimental for health;
Compositions 1A, 1B, 1C and 1D of Example 1 inhibited S. epidermis biofilm formation to a lesser extent compared to Biolin/P, Biotilys and Senseryn formulations. As mentioned above, inhibition of S. epidermidis biofilm growth while promoting S. aureus biofilm growth (FIG. 3) is detrimental to the proper balance of the epidermal microbiome in dermatoses.

### Example 3: Lipid Nourishing Cream (Night Cream)

In Example 3 a composition according to the invention of the following composition was used:

| | Composition 1E |
|---|---|
| | [% by weight] |
| BIOLIN/P | 0.10 |
| BIOTILYS | 0.10 |
| SENSERIN | 2.00 |

Which, when converted to the content of pure active ingredients, results in the following composition:

| | Composition 1E |
|---|---|
| | [% by weight] |
| Inulin (INCI: Inulin) | 0.08 |
| Alpha-glucan (INCI: Alpha-Glucan Oligosaccharide) | 0.02 |
| Lysate of a fermentation product of bacteria of the | |
| Lactobacillus genus (INCI: Lactobacillus ferment | |
| lysate) | 0.0058 |
| Common hops extract | |
| (INCI: Humulus Lupulus cone extract) | 0.04 |

An aqueous phase was prepared by mixing glycerol, hydroxyacetophenone (SYMSAVE H, Symrise) and purified water together in a mixer, followed by heating the mixture to 75° C. and stirring until the components are dissolved. Carbomer (CARBOPOL ULTREZ 30 POLYMER, Lubrizol) was added to the mixture and the whole mixture was stirred for 5 min at 1500 rpm. The previously prepared aqueous sodium carbonate solution was then added to the mixture and mixing was continued for 5 min at 1500 rpm. In a separate mixer, an oil phase was prepared by mixing together a mixture of cetostearyl alcohol and cetostearyl alcohol polyglucoside (MONTANOV 68, Seppic), a mixture of arachidyl alcohol, behenyl alcohol and arachidyl alcohol polyglucoside (MONTANOV 202, Seppic), candelilla wax, shea butter, jojoba oil, rapeseed oil, pentaerythritol tetraisostearate (PRISORINE 3631, Croda), hydrogenated polydecene (NEXBASE 2008, Neste Oil), a mixture of oleyl erucinate, tocopherol and citrate of hydrogenated vegetable oil monoglycerides (CETIOL J600, BASF), a mixture of vegetable oil, hydrogenated vegetable oil and candelilla wax (CEGESOFT VP, BASF). The resulting mixture was heated to 75° C. and stirred. The water phase and the oil phase were combined and homogenized for 7 min at 1500 rpm. After mixing, the mixture was cooled to 40° C. The previously prepared aqueous solution of niacinamide was then added to the mixture and the whole mixture was homogenized for 5 min at 1000 rpm. The resulting mixture was cooled to 35° C. Subsequently, the components of composition 1E and an aliquot of water were added to the mixture and the whole was stirred for 3 min at 1000rpm. 1,2-hexanediol (HYDROLITE 6, Symrise) was added to the resulting mixture and the whole mixture was stirred for 3 min at 1200 rpm. The resulting mixture was cooled to 25° C. A lipid nourishing cream composition of the following composition was obtained:

| | |
|---|---|
| 2.00% by weight | of glycerol (INCI: Glycerin), |
| 0.80% by weight | of hydroxyacetophenone (INCI: |
| | Hydroxyacetophenone), |
| 0.10% by weight | of carbomer (INCI: Carbomer), |
| 0.04% by weight | of sodium carbonate (INCI: Sodium Carbonate), |
| 2.00% by weight | of a mixture of cetostearyl alcohol and |
| | cetostearyl alcohol polyglucoside (INCI: |
| | Cetearyl Alcohol & Cetearyl Glucoside), |
| 2.00% by weight | of a mixture of arachidyl alcohol, behenyl |
| | alcohol and arachidyl alcohol polyglucoside |
| | (INCI: Arachidyl Alcohol & Behenyl |
| | Alcohol & Arachidyl Glucoside), |
| 0.20% by weight | of candelilla wax (INCI: Candelilla Cera), |
| 3.00% by weight | of shea butter (INCI: Shea Butter), |
| 1.00% by weight | of jojoba oil (INCI: Simmondsia Chinensis |
| | Seed Oil), |
| 5.00% by weight | of rapeseed oil (INCI: Canola Oil), |
| 1.50% by weight | of pentaerythritol tetraisostearate (INCI: |
| | Pentaerythrityl Tetraisostearate), |
| 5.00% by weight | of hydrogenated polydecene (INCI: |
| | Hydrogenated Polydecene), |
| 2.00% by weight | of a mixture of oleyl erucate, tocopherol and |
| | citrate of hydrogenated vegetable oil |
| | monoglycerides (INCI: Oleyl Erucate & |
| | Tocopherol & Hydrogenated Vegetable |
| | Glycerides Citrate), |
| 1.00% by weight | of a mixture of vegetable oil, hydrogenated |
| | vegetable oil and candelilla wax |
| | (INCI: Olus Oil & Hydrogenated |
| | Vegetable Oil & Candelilla Cera), |
| 2.00% by weight | of niacinamide (INCI: Niacinamide), |
| 2.00% by weight | of a mixture of propanediol, glycerol, |
| | common hops extract and citric acid |
| | (INCI: Propanediol & Glycerin & Humulus |
| | Lupulus (Hops) Extract & Citric Acid), |
| 0.10% by weight | of a mixture of purified water, lysate of a |
| | fermentation product of bacteria of the |
| | Lactobacillus genus, sodium benzoate and |
| | potassium sorbate (INCI: Aqua & Lactobacillus |
| | Ferment Lysate & Sodium Benzoate & |
| | Potassium Sorbate), |
| 0.10% by weight | of a mixture of inulin and alpha-glucan (INCI: |
| | Inulin & Alpha-Glucan Oligosaccharide), |
| 1.50% by weight | of 1,2-hexanediol (INCI: 1,2-Hexanediol), |
| up to 100% by weight | of purified water. |

### Example 4: Face Toner

In Example 4 a composition according to the invention of the following composition was used:

| | Composition 1F |
|---|---|
| | [% by weight] |
| BIOLIN/P | 0.10 |
| BIOTILYS | 0.05 |
| SENSERIN | 0.2 |

Which, when converted to the content of pure active ingredients, results in the following composition:

| | Composition 1F |
|---|---|
| | [% by weight] |
| Inulin (INCI: Inulin) | 0.08 |
| Alpha-glucan (INCI: Alpha-Glucan Oligosaccharide) | 0.02 |
| Lysate of a fermentation product of bacteria of the | 0.0029 |
| Lactobacillus genus | |
| (INCI: Lactobacillus ferment lysate) | |
| Common hops extract | 0.004 |
| (INCI: Humulus Lupulus cone extract) | |

An aqueous phase was prepared by mixing propanediol (ZEMEA, CovationBio), a mixture of gluconolactone, sodium benzoate and calcium gluconate (GEOGARD ULTRA, Arxada), sodium citrate and purified water together in a mixer, followed by heating the mixture to 65° C. An aliquot of water was introduced into the mixture and stirring was continued. The components of Composition 1F and an aliquot of water were then added to the mixture one at a time every 2 min, without stopping the stirring. A mixture of seawater and glycerol was introduced into the resulting mixture and the whole mixture was stirred for 2 min. A face toner composition of the following composition was obtained:

| | |
|---|---|
| 2.00% by weight | of propanediol (INCI: Propanediol), |
| 0.60% by weight | of a mixture of gluconolactone, sodium benzoate and calcium |
| | gluconate (INCI: Gluconolactone & Sodium Benzoate & Calcium |
| | Gluconate), |
| 0.20% by weight | of a mixture of propanediol, glycerol, common hops extract and |
| | citric acid (INCI: Propanediol & Glycerin & Humulus Lupulus |
| | (Hops) Extract & Citric Acid), |
| 0.05% by weight | of a mixture of purified water, lysate of a fermentation product of |
| | bacteria of the Lactobacillus genus, sodium benzoate and |
| | potassium sorbate (INCI: Aqua & Lactobacillus Ferment Lysate & |
| | Sodium Benzoate & Potassium Sorbate), |
| 0.10% by weight | of a mixture of inulin and alpha-glucan (INCI: Inulin & Alpha- |
| | Glucan Oligosaccharide), |
| 0.10% by weight | of a mixture of sea water and glycerol (INCI: Maris Aqua (Sea |
| | Water) & Glycerin), |
| up to 100% by weight | of purified water. |

### Example 5: Ceramide Anti-Wrinkle Cream (Night and Day Cream)

In Example 5 a composition according to the invention of the following composition was used:

| | Composition 1G |
|---|---|
| | [% by weight] |
| BIOLIN/P | 1.00 |
| BIOTILYS | 0.20 |
| SENSERIN | 2.00 |

Which, when converted to the content of pure active ingredients, results in the following composition:

| | Composition 1G |
|---|---|
| | [% by weight] |
| Inulin (INCI: Inulin) | 0.8 |
| Alpha-glucan (INCI: Alpha-Glucan Oligosaccharide) | 0.2 |
| Lysate of a fermentation product of bacteria of the | 0.0116 |
| Lactobacillus genus | |
| (INCI: Lactobacillus ferment lysate) | |
| Common hops extract | 0.04 |
| (INCI: Humulus Lupulus cone extract) | |

An aqueous phase was prepared by mixing propanediol (ZEMEA PROPANEDIOL, CovationBio), pentylene glycol (HYDROLITE 5, Symrise), hydroxyacetophenone (Symsave H, Symrise), phytic acid sodium salt (DERMOFEEL PA-12, Evonik), glycerol and an aliquot of purified water together in a mixer, followed by heating the mixture to 80° C. An aqueous solution of carbomer (CARBOPOL ULTREZ 10, Lubrizol) was added to the mixture and the whole mixture was stirred for 10 min at 1500 rpm. The previously prepared aqueous sodium carbonate solution was then added to the mixture and stirring was continued for 3 min at 1500 rpm. Potassium cetyl phosphate (AMPHISOL K, DSM) was added to the aqueous phase and the mixture was homogenized for 5 min at 800 rpm. In a separate mixer, an oil phase was prepared by mixing together a mixture of sorbitol olivate and cetostearyl olivate (OLIVEM 1000, Hallstar), cetostearyl alcohol, glycerol stearate (CUTINA GMS, BASF), a mixture of esters of caprylic and capric acids with coconut oil alcohols and tocopherol (CETIOL C5, BASF), isopropyl isostearate (PRISORINE 2021, Croda), dibutyl adipate (CETIOL B, BASF), shea butter, 2-[4-(diethylamino)-2-hydrokybenzoyl] benzoic acid hexyl ester (UVINUL A PLUS, BASF), butyl octyl salicylate (HALLBRITE BHB, Hallstar), bemotrizinol (TINOSORB S,

BASF), ethylhexyl triazone (UVINUL T-150, BASF) and a mixture of dioctyl carbonate and tocopherol (CETIOL CC, BASF). The resulting mixture was heated to 80° C. and then a mixture of tocopherol and sunflower oil (COSPHADERM T-70 NON GMO, Cosphatec), moringa oil and cottonseed oil was added and mixed. The aqueous phase and oil phase were combined and homogenized for 7 min at 1500 rpm. After mixing, the mixture was cooled to 60° C. A mixture of bisoctrizole, purified water and decyl glucoside (TINOSORB M, BASF) was added to the mixture and the whole mixture was stirred for 5 min at 1500 rpm. After mixing, the mixture was cooled to 35° C. A mixture of (9Z)-N-[(2S,3S,4R)-1,3,4-trihydroxyoctadecan-2-yl]octadec-9-enamide, 2-hydroxy-N-[(2S,3S,4R)-1,3,4-trihydroxy-2-octadecanyl]octadecanamide, 30-oxo-30-{[(25,3S,4R)-1,3,4-trihydroxy-2-octadecanyl]amino}triacontyl-(9Z,12Z)-9,12-octadecadienoate, phytosphingosine, cholesterol, behenic acid, polyglycerol-10 stearate, polyglycerol-6 behenate, cetostearyl alcohol, glycerol stearate, glycerol, cetostearyl sulfate sodium salt, triethyl citrate, sodium levulinate and potassium sorbate (SK-INFLUX EVOLVE MB, Evonik) and purified water was added to the mixture and the whole mixture was stirred for 2 min at 1000 rpm./min. Subsequently, the components of Composition G and an aliquot of water were added to the mixture and the whole mixture was stirred for 3 min at 1200 rpm. The resulting mixture was cooled to 25° C. A ceramide anti-wrinkle cream composition of the following composition was obtained:

| | |
|---|---|
| 3.00% by weight | of propanediol (INCI: Propanediol), |
| 2.50% by weight | of pentylene glycol (INCI: Pentylene Glycol), |
| 0.40% by weight | of hydroxyacetophenone (INCI: Hydoxyacetophenone), |
| 0.10% by weight | of phytic acid sodium salt (INCI: Sodium Phytate), |
| 3.00% by weight | of glycerol (INCI: Glycerin), |
| 0.30% by weight | of carbomer (INCI: Carbomer), |
| 0.10% by weight | of sodium carbonate (INCI: Sodium Carbonate), |
| 2.00% by weight | of potassium cetyl phosphate (INCI: Potassium Cetyl |
| | Phosphate), |
| 0.80% by weight | of a mixture of sorbitol olivate and cetostearyl olivate (INCI: |
| | Cetearyl Olivate & Sorbitan Olivate), |
| 1.80% by weight | of cetostearyl alcohol (INCI: Cetearyl Alcohol), |
| 0.80% by weight | of glycerol stearate (INCI: Glyceryl Stearate), |
| 4.00% by weight | of a mixture of esters of caprylic and capric acids with |
| | coconut oil alcohols and tocopherol (INCI: Coco-Caprylate & |
| | Tocopherol), |
| 3.00% by weight | of isopropyl isostearate (INCI: Isopropyl Isostearatel), |
| 2.00% by weight | of dibutyl adipate (INCI: Dibudyl Adipate), |
| 1.50% by weight | of shea butter (INCI: Shea Butter), |
| 2.00% by weight | of 2-[4-(diethylamino)-2-hydroxybenzoyl]benzoic acid hexyl |
| | ester (INCI: Diethylamino Hydroxybenzoyl Hexyl Benzoate), |
| 2.00% by weight | of butyl octyl salicylate (INCI: Butyloctyl Salicylate), |
| 0.80% by weight | of bemotrizinol, (INCI: Bis-Ethylhexyloxyphenol |
| | Methoxyphenyl Triazine), |
| 0.80% by weight | of ethylhexyl triazone (INCI: Ethylhexyl Triazone), |
| 2.00% by weight | of a mixture of dioctyl carbonate and tocopherol (Dicaprylyl |
| | Carbonate & Tocopherol), |
| 0.10% by weight | of a mixture of tocopherol and sunflower oil (INCI: |
| | Tocopherol & Helianthus Annuus (Sunflower) Seed Oil), |
| 0.10% by weight | of moringa oil (INCI: Moringa Oil), |
| 1.00% by weight | of cottonseed oil (INCI: Gossypium (Cotton) Seed Oil), |
| 2.00% by weight | of a mixture of bisoctrizole, purified water and decyl |
| | glucoside (INCI: Methylene Bis-Benzotriazolyl |
| | Tetramethylbutylphenol (nano) & Aqua & Decyl Glucoside), |
| 1.00% by weight | of mixtures of (9Z)-N-[(2S,3S,4R)-1,3,4- |
| | trihydroxyoctadecan-2-yl]octadec-9-enamide, 2-hydroxy-N- |
| | [(2S,3S,4R)-1,3,4-trihydroxy-2- |
| | octadecanyl]octadecanamide, 30-oxo-30-{[(25,3S,4R)- |
| | 1,3,4-trihydroxy-2-octadecanyl]amino}triacontyl-(9Z,12Z)- |
| | 9,12-octadecadienoate, phytosphingosine, cholesterol, |
| | behenic acid, polyglycerol-10 stearate, polyglycerol-6 |
| | behenate, cetostearyl alcohol, glyceryl stearate, glycerol, |
| | cetostearyl sulfate sodium salt, triethyl citrate, sodium |
| | levulinate and potassium sorbate (INCI: Ceramide NP & |
| | Ceramide AP & Ceramide EOP & Phytosphingosine & |
| | Cholesterol & Behenic Acid & Polyglyceryl-10 Stearate & |
| | Polyglyceryl-6 Behenate & Cetearyl Alcohol & Glyceryl |
| | Stearate & Glycerin & Sodium Cetearyl Sulfate & Triethyl |
| | Citrate & Sodium Levulinate & Potassium Sorbate), |
| 2.00% by weight | a mixture of propanediol, glycerol, common hops extract and |
| | citric acid (INCI: Propanediol & Glycerin & Humulus Lupulus |
| | (Hops) Extract & Citric Acid), |
| 0.20% by weight | a mixture of purified water, lysate of a fermentation product |
| | of bacteria of the Lactobacillus genus, sodium benzoate and |
| | potassium sorbate (INCI: Aqua & Lactobacillus Ferment |
| | Lysate & Sodium Benzoate & Potassium Sorbate), |
| 1.00% by weight | a mixture of inulin and alpha-glucan (INCI: Inulin & Alpha- |
| | Glucan Oligosaccharide). |
| up to 100% by weight | purified water. |

### Example 6: Application Studies

### Description of the Studies

Using a lipid nourishing cream (Example 3), a face toner (Example 4) and a ceramide anti-wrinkle cream (Example 5), application studies were carried out with 25 people aged 21 to 75 years, 24 people aged 26 to 72 years and 24 people aged 27 to 74 years with different skin types, respectively, as shown in Table 5.

TABLE 5
Skin types in participants of application studies

| | Skin type | | |
|---|---|---|---|
| | | Night and | |
| | Night cream | day cream | Face toner |
| | (Example 3) | (Example 5) | (Example 4) |
| | Number of volunteers/age | | |
| | 25 / 21-75 years | 24 / 27-74 years | 24 / 26-72 years |
| | (23 females, | (24 females, | (23 female, |
| | 2 men) | 1 male) | 1 male) |
| | % of respondents | | |
| sensitive | 96% | 63% | 74% |
| normal | 0% | 17% | 0% |
| fat | 0% | 0% | 4% |
| hyperreactive | 32% | 0% | 35% |
| mixed | 36% | 33% | 43% |
| vascular | 64% | 25% | 61% |
| dry | 36% | 46% | 39% |
| allergic | 40% | 29% | 39% |

Prior to the start of the study participants were qualified using a sensitivity scale, which was completed by the volunteer prior to the start of the study (according to Legeas C, Misery L, Fluhr JW, Roudot AC, Ficheux AS, Brenaut E. Proposal for Cut-off Scores for Sensitive Skin on Sensitivity Scale-10 in a Group of Adult Women. Acta Derm Venereol. 2021 Jan. 13; 101 (1); L. Misery, C. Jean-Decoster, S. Mery, V. Georgescu, V. Sibaud, A New Ten-Item Questionnaire For Assessing Sensitive Skin: the Sensitive Scale-10, Acta Derm Venereol 2014, 94, 635-639).

The compositions of the example were applied according to the manufacturer's instructions. The night cream and the night and day cream were applied daily in the morning and/or evening to the cleansed face skin, while the toner was used to rinse the face after using the cleansing cosmetics. At the end of the four-week application study, study participants evaluated the skincare properties of the tested product and expressed their subjective impressions in individual surveys. None of the tested products caused adverse reactions such as watering eyes, flaky skin, burning and stinging sensation of the skin, rash, redness of the skin, pruritus, itchy skin, pimples, papules, urticarial blisters, or caused general discomfort in the study participants.

### Results

### Night Cream (Example 3)

According to the results of the post-application survey, study participants assessed that the tested product (% values refer to the number of participants the parameter applies to):
effectively soothes the skin (a parameter confirmed by 96%),
reduces/soothes skin irritation (a parameter confirmed by 91% of participants),
reduces skin redness (a parameter confirmed by 79% of participants the feature refers to),. reduces the sensation of burning on the skin (a parameter confirmed by 75% of participants),
minimizes skin discomfort (a parameter confirmed by 88% of participants),
reduces symptoms of skin hypersensitivity (a parameter confirmed by 75% of participants),
reduces skin dryness (a parameter confirmed by 88% of participants),
reduces skin flaking (a parameter confirmed by 81% of participants),
reduces skin roughness (a parameter confirmed by 90% of participants),
minimizes the stinging sensation on the skin (a parameter confirmed by 72% of participants),
reduces redness and discomfort caused by dry skin (a parameter confirmed by 91% of participants),
protects the skin from irritants and adverse external factors: wind, rain, frost (a parameter confirmed by 84% of participants),
shows a strong soothing and calming effect (a parameter confirmed by 88% of participants),
protects the skin from irritation (a parameter confirmed by 80% of participants),
protects against skin dehydration (a parameter confirmed by 76% of participants),

Study participants also rated their skin after one application of the cosmetic as:
hydrated (a parameter confirmed by 92% of participants),
nourished (a parameter confirmed by 76% of participants),
smoothed (a parameter confirmed by 84% of participants),
lubricated (a parameter confirmed by 72% of participants),
soothed, relieved (a parameter confirmed by 84% of participants),
soft to the touch (a parameter confirmed by 96% of participants),
flexible (parameter confirmed by 84% of participants),
regenerated (a parameter confirmed by 72% of participants).

In addition, the studies showed a 69% improvement in skin hydration and a 68% improvement in skin smoothness (based on an analogue scale where 0-no effect, 100-intense effect). A 60% reduction in skin sensitivity symptoms was also observed in 96% of study participants (according to Legeas C, Misery L, Fluhr JW, Roudot AC, Ficheux AS, Brenaut E. Proposal for Cut-off Scores for Sensitive Skin on Sensitive Scale-10 in a Group ofAdult Women. Acta Derm Venereol. 2021 Jan. 13;101 (1); L. Misery, C. Jean-Decoster, S. Mery, V. Georgescu, V. Sibaud, A New Ten-Item Questionnaire For Assessing Sensitive Skin: the Sensitive Scale-10, Acta Derm Venereol 2014, 94, 635-639).

### Face Toner (Example 4)

According to the results of the post-application survey, study participants assessed that the tested product (% values refer to the number of participants the parameter applies to):
effectively soothes the skin (parameter confirmed by 96% of participants),
reduces/soothes skin irritation (parameter confirmed by 82% of participants).

Study participants also rated their skin immediately after using the cosmetic as:
hydrated (a parameter confirmed by 83% of participants),
nourished (a parameter confirmed by 70% of participants),
soothed, relieved (a parameter confirmed by 78% of participants),
soft to the touch (a parameter confirmed by 87% of participants),
flexible (a parameter confirmed by 65% of participants).

A 72% reduction in skin sensitivity symptoms was also observed in 95% of study participants reporting the parameter.

### Night and Day Cream (Example 5)

According to the results of the post-application survey, participants in the study assessed that the product under study (% values refer to the number of participants affected by the parameter):
effectively soothes the skin (a parameter confirmed by 90% of participants),
reduces/soothes skin irritation (a parameter confirmed by 81% of participants),
reduces skin redness (a parameter confirmed by 56% of participants),
reduces the sensation of burning on the skin (a parameter confirmed by 71% of participants),
minimizes skin discomfort (a parameter confirmed by 78% of participants),
reduces symptoms of skin hypersensitivity (a parameter confirmed by 68% of participants),
reduces skin dryness (a parameter confirmed by 71% of participants),
reduces skin roughness (a parameter confirmed by 86% of participants),
reduces redness and discomfort caused by dry skin (a parameter confirmed by 63% of participants),
shows a strong soothing and calming effect (parameter confirmed by 73% of participants),
protects the skin from irritation (parameter confirmed by 68% of participants).

Study participants also rated their skin after one application of the cosmetic as:
hydrated (a parameter confirmed by 92% of participants),
nourished (a parameter confirmed by 83% of participants),
smoothed (a parameter confirmed by 79% of participants),
lubricated (a parameter confirmed by 71% of participants),
soothed, relieved (a parameter confirmed by 79% of participants),
soft to the touch (a parameter confirmed by 83% of participants),
flexible (a parameter confirmed by 67% of participants),
regenerated (a parameter confirmed by 75% of participants).

In addition, the studies showed a 65% improvement in skin smoothness (based on an analogue scale, where 0—no effect, 100—intense effect). A reduction in skin sensitivity symptoms by 80% was also observed in 65% of study participants reporting the parameter.

1. A composition comprising inulin, alpha-glucan, common hops extract and lysate of a fermentation product of bacteria of the Lactobacillus genus.

2. The composition according to claim 1, characterized in that it comprises:

| | |
|---|---|
| 0.008 to 0.8% by weight | of inulin, |
| 0.002 to 0.2% by weight | of alpha-glucan, |
| 0.00002 to 0.04% by weight | of common hops extract, |
| 0.000058 to 0.0116% by weight | of lysate of a fermentation product of |
| | bacteria of the Lactobacillus genus, |

and at least one acceptable excipient in addition to 100% by weight, the percentages being expressed based on the total weight of the composition.

3. The cosmetic composition according to claim 1, wherein it comprises

| | |
|---|---|
| 0.008 to 0.08% by weight | of inulin, |
| 0.002 to 0.02% by weight | of alpha-glucan, |
| 0.00002 to 0.002% by weight | of common hops extract, |
| 0.000058 to 0.0058% by weight | of lysate of a fermentation product of |
| | bacteria of the Lactobacillus genus, |

the percentages being expressed based on the total weight of the composition.

4. The cosmetic composition according to claim 1, wherein it comprises

| | |
|---|---|
| 0.08 to 0.8% by weight | of inulin, |
| 0.02 to 0.2% by weight | of alpha-glucan, |
| 0.004 to 0.04% by weight | of common hops extract, |
| 0.0029 to 0.0116% by weight | of lysate of a fermentation product of |
| | bacteria of the Lactobacillus genus, |

the percentages being expressed based on the total weight of the composition.

5. A cosmetic product comprising the composition as defined in claim 1.

6. The cosmetic product according to claim 5, wherein it comprises from 0.05 to 4.00% by weight of the composition as defined in claim1 in relation to the total weight of the cosmetic product and at least one additional cosmetically acceptable active agent and/or at least one additional cosmetically acceptable excipient.

7. The cosmetic product according to claim 5, wherein it is in liquid, solid or semi-solid form, for example in the form of an emulsion, including an O/W emulsion and an moisturizing O/W emulsion, a gel, a foam, or a solution.

8. The cosmetic product according to claim 5, wherein it is selected from a cream, including an under-eye cream or a night cream, a day cream, a skin cleansing product, e.g. for the face skin, including a gel, a foam, a micellar liquid, a milk, a gel serum, a toner, preferably a face toner, an oil, e.g. a face oil.

9. The cosmetic product according to claim 5, wherein it comprises

| | |
|---|---|
| 0.05 to 4.00% by weight | of the composition as defined, |
| 0.00 to 22.00% by weight | at least one emollient and/or wax, |
| 0.00 to 5.00% by weight | of at least one emulsifying agent, |
| 0.01 to 16.00% by weight | of at least one moisture-binding agent, |
| 0.001 to 5.00% by weight | of at least one additional cosmetically |
| | acceptable active agent, |
| 0.01 to 1.50% by weight | of at least one preservative, |
| 0.001 to 1.80% by weight | of at least one cosmetically acceptable |
| | auxiliary substance, |
| 0.00 to 3.00% by weight | of at least one consistency adjusting agent, |
| 0.00 to 0.30% by weight | of at least one fragrance composition, |
| 0.00 to 21.00% by weight | of at least one UV filter, |
| 0.00 to 14.00% by weight | of at least one surfactant, |
| up to 100% by weight | of purified water. |

10. The cosmetic product according to claim 5, characterized in that it is in the form of an o/w moisturizing emulsion, in particular a night face cream or an eye cream comprising:

| | |
|---|---|
| 0.05 to 4.00% by weight | of the composition as defined |
| 5.00 to 19.00% by weight | of at least one emollient and/or wax, |
| 0.50 to 4.00% by weight | of at least one emulsifying agent, |
| 0.01 to 5.00% by weight | of at least one moisture-binding agent, |
| 0.001 to 5.00% by weight | of at least one additional cosmetically |
| | acceptable active agent, |
| 0.01 to 1.50% by weight | of at least one preservative, |
| 0.001 to 0.50% by weight | of at least one cosmetically auxiliary |
| | substance, |
| 0.01 to 1.50% by weight | of at least one consistency adjusting agent, |
| 0.01 to 0.30% by weight | of at least one fragrance composition, |
| up to 100% by weight | of purified water. |

11. The cosmetic product according to claim 5, characterized in that it is in the form of an o/w emulsion, in particular a day face cream comprising:

| | |
|---|---|
| 0.05 to 4.00% by weight | the composition as defined, |
| 5.00 to 22.00% by weight | of at least one emollient and/or wax, |
| 0.50 to 4.00% by weight | of at least one emulsifying agent, |
| 0.01 to 7.00% by weight | of at least one moisture-binding agent, |
| 1.00 to 21.00% by weight | of at least one UV filter, |
| 0.001 to 4.00% by weight | of at least one additional cosmetically |
| | acceptable active agent, |
| 0.01 to 1.50% by weight | of at least one preservative, |
| 0.001 to 1.00% by weight | of at least one cosmetically acceptable |
| | auxiliary substance, |
| 0.01 to 3.00% by weight | of at least one consistency adjusting agent, |
| up to 100% by weight | of purified water. |

12. The cosmetic product according to claim 5, characterized in that it is in the form of a face cleansing product, in particular a gel, foam, a micellar liquid or a gel serum for the face comprising:

| | |
|---|---|
| 0.05 to 4.00% by weight | of the composition as defined, |
| 0.01 to 16.00% by weight | of at least one moisture-binding agent, |
| 1.00 to 14.00% by weight | of at least one surfactant, |
| 0.01 to 1.80% by weight | of at least one additional cosmetically |
| | acceptable active agent, |
| 0.01 to 1.50% by weight | of at least one preservative, |
| 0.001 to 1.80% by weight | of at least one cosmetically acceptable |
| | auxiliary substance, |
| 0.01 to 0.90% by weight | of at least one consistency adjusting agent, |
| 0.01 to 0.30% by weight | of at least one fragrance composition, |
| up to 100% by weight | of purified water. |

13. The cosmetic product according to claim 5, characterized in that it is in the form of a face cleansing product, in particular a face cleansing milk comprising:

| | |
|---|---|
| 0.05 to 4.00% by weight | of the composition as defined, |
| 5.00 to 15.00% by weight | of at least one emollient, |
| 1.00 to 5.00% by weight | of at least one emulsifying agent, |
| 0.01 to 7.00% by weight | of at least one moisture-binding agent, |
| 0.01 to 1.00% by weight | of at least one surfactant, |
| 0.001 to 1.00% by weight | of at least one additional cosmetically |
| | acceptable active agent, |
| 0.01 to 1.50% by weight | of at least one preservative, |
| 0.001 to 0.50% by weight | of at least one cosmetically acceptable |
| | auxiliary substance, |
| 0.01 to 1.00% by weight | of at least one consistency adjusting agent, |
| up to 100% by weight | of purified water. |

14. The cosmetic product according to claim 5, characterized in that it is in the form of a face toner comprising:

| | |
|---|---|
| 0.05 to 4.00% by weight | of the composition as defined, |
| 0.01 to 2.00% by weight | of at least one moisture-binding agent, |
| 0.001 to 1.00% by weight | of at least one additional cosmetically |
| | acceptable active agent, |
| 0.01 to 1.50% by weight | of at least one preservative, |
| 0.01 to 1.00% by weight | of at least one cosmetically acceptable |
| | auxiliary substance, |
| up to 100% by weight | of purified water. |
